(19) United States
(12) Patent Application Publication
(10) Pub. No.: US 2025/0336551 A1
(43) Pub. Date: Oct. 30, 2025

(54) METHODS AND SYSTEMS FOR ACTIVE LEARNING FOR HEALTH EQUITY

(71) Applicant: Lauren Jan Kopsick, Milford, CT (US)

(72) Inventor: Lauren Jan Kopsick, Milford, CT (US)

(21) Appl. No.: 19/184,227

(22) Filed: Apr. 21, 2025

Related U.S. Application Data
(60) Provisional application No. 63/639,957, filed on Apr. 29, 2024.

(51) Int. Cl. G16H 80/00; G16H 20/00; G16H 40/00

(52) U.S. Cl. CPC G16H 80/00; G16H 20/00; G16H 40/00

(57) ABSTRACT

The described technology pertains to educational advancements, specifically methods and systems for active learning and self-advocacy training in healthcare navigation. The present invention involves a novel training method that integrates interdisciplinary social and behavior change communication within a collaborative learning environment. This method utilizes storytelling, live examples, and practical exercises, incorporating five principles of self-advocacy individuals to navigate the healthcare system(s). The system includes modular training components covering basic healthcare navigation, mental health, pharmacy interactions, specialty healthcare, and hospital care. Principal uses include training youth transitioning to adult care, employee wellness programs, and community health initiatives. The described technology can be implemented in various environments and is adaptable to different learning styles, fostering a culture of empowerment and health equity.

### CROSS-REFERENCE TO RELATED APPLICATIONS

This application claims benefit to Provisional Application No. 63/639,957, filed Apr. 29, 2024, the contents of which are herein incorporated by reference.

### BACKGROUND OF THE INVENTION

### Field of Endeavor

The present disclosure pertains to educational technologies, specifically methods and systems for active learning and self-advocacy training in healthcare navigation.

### Background of Related Art

The healthcare system in the United States is notoriously complex, often requiring individuals to navigate a labyrinth of procedures and policies to access necessary services. This complexity is compounded by a general lack of health literacy among the population, with a significant portion of youth and adults struggling to understand and effectively engage with the healthcare system. Many individuals, particularly those from underserved communities, seniors, and non-native language speakers, face additional barriers due to standardized training programs that do not cater to diverse learning needs or promote self-advocacy. These programs often focus on compliance rather than empowering individuals to navigate the system independently.

Existing educational methods in healthcare navigation often rely on passive approaches, such as lectures or reading materials, which do not actively engage learners or address the specific challenges faced by different populations. These methods do not sufficiently equip individuals with the life skills needed, such as communication and decision-making, to advocate for themselves effectively.

There is a pressing need for innovative training and learning methods that foster active participation and are adaptable to various learning styles, empowering individuals to take control of their healthcare journeys.

### SUMMARY OF THE INVENTION

In one embodiment, the disclosure provides a method for training and self-advocacy comprising providing one or more narratives to a user, performing one or more learning modules configured to teach one or more outcomes, performing one or more real-world exercises based on the learning modules, and sharing the one or more outcomes with at least one additional user.

In another embodiment, the real-world exercises comprise a live telephone conversation with an individual identified in the learning modules.

In further embodiments, the learning modules include at least one module selected from a group comprising a basic healthcare navigation module, a mental health toolbox module, a pharmacy education module, a specialty healthcare module, or an advanced healthcare navigation module.

### BRIEF DESCRIPTION OF THE DRAWINGS

FIG. 1 is a flow chart diagram illustrating the method for training and active learning in healthcare advocacy;

FIG. 2 is a flow chart diagram illustrating the Five Principles of Self-Advocacy within the training and learning method; and

FIG. 3 is a block diagram illustrating the system's modular approach to healthcare navigation and advocacy training.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

The healthcare system in the United States is notoriously complex, often requiring individuals to navigate a labyrinth of procedures, policies, and terminologies to access necessary services. This complexity is compounded by the fact that a significant portion of the population lacks the necessary health literacy to effectively advocate for themselves within this system. Studies indicate that 85% of youth transitioning to adult healthcare are unprepared to manage their own health needs, and 9 out of 10 adults have limited health literacy. This lack of understanding and preparedness can lead to inadequate healthcare access and poor health outcomes, particularly among underserved communities, seniors, youth, immigrants, and non-native language speakers.

Existing training programs aimed at improving health literacy and self-advocacy skills are often insufficient. These programs tend to be standardized, focusing on compliance rather than empowerment, and are typically delivered in passive learning environments such as lectures or computer-based modules. Such methods assume a one-size-fits-all approach to learning, which fails to account for the diverse educational backgrounds, socioeconomic situations, and learning styles of individuals. Moreover, these programs often lack practical components that teach important life skills such as communication, decision-making, and coping strategies, which are necessary for effective self-advocacy.

The present approach addresses these shortcomings by providing a novel training and learning method that integrates interdisciplinary social and behavior change communication within a collaborative and active learning environment.

This method is designed to empower individuals by teaching them how to navigate the healthcare system effectively and advocate for themselves. The training incorporates five specific principles of self-advocacy, tailored to the needs of different populations, and can be facilitated in both live and virtual interactive environments. By engaging participants in active learning through storytelling, live examples, and practical exercises, the approach aims to replace fear with empowerment, enabling individuals to become confident self-advocates. The method also includes a train-the-trainer model, allowing participants to become trainers themselves and extend the program's reach into their communities, thereby fostering a culture of empowerment and health equity.

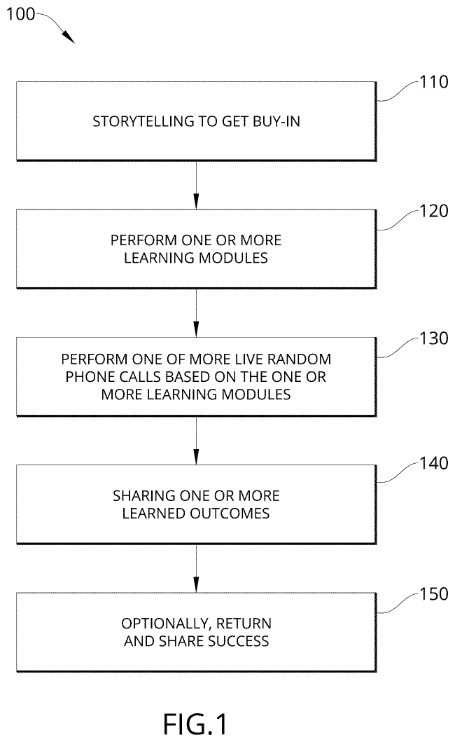

Referring now to FIG. 1, a training and self-advocacy method 100 of the present invention is illustrated. Briefly, and described in more detail below, training and self-advocacy method 100 is based on storytelling in an active learning environment (discussion, practice, teach another person to retain 90% of knowledge) and includes: life skills communication, decision-making, and coping strategies, making live calls with participants to get buy-in, and sharing outcomes to cement learning.

Method 100 begins at step 110 where one or more facilitator(s) can tell a story or ask a question to the one or more participant(s) thereby requiring the one or more participants to exercise cognitive abilities. In embodiments, the story, or question, is configured to allow the one or more participants to access their long term memory, such as a contemplative question, or a question about an event in their history. Alternatively, the story, or question, is configured to give the one or more participants context into learning, such as am explanation of personality types, i.e. think-first personality vs. talk-first personality. Advantageously, telling a story to the one or more participant(s) achieves buy-in with the one or more participant(s) thereby improving learning outcomes.

At step 120, once buy-in is established by the one or more facilitator(s) one or more modules 310-350 of Learning Module System 300 are performed.

At step 130, once the one or more modules 310-350 are performed, the one or more participant(s) can perform one or more phone calls. In embodiments, the one or more phone calls can be based on information learned during step 120. Additionally, the one or more phone calls can be performed lived, with the one or more facilitator(s), and one or more participant(s) participating in-whole, or in-part. In embodiments, the one or more participant(s) are taught one or more questions to ask during the one or more modules 310-350, which are HIPAA compliant questions.

At step 140, after the one or more phone calls are performed, the one or more participant(s) can share one or more learning outcomes with one or more additional person(s), such as additional participants, facilitator(s), or other person(s).

At step 150, optionally, the one or more participant(s) can return and share one or more successes achieved as a result of utilizing method 100.

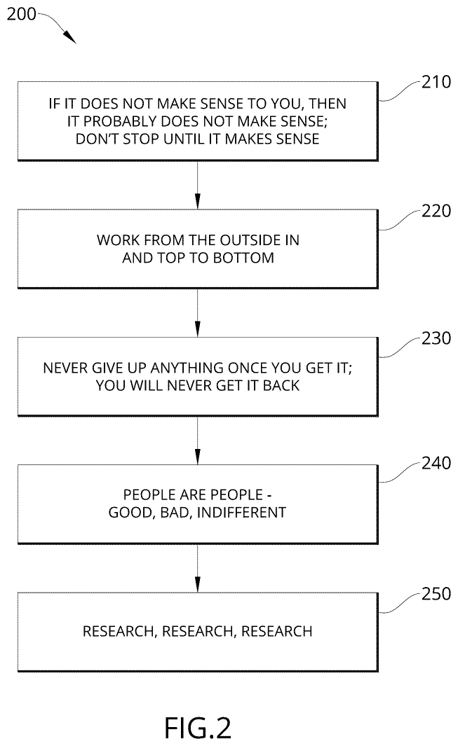

Referring now to FIG. 2, the Five Principles of Self-Advocacy 200 are illustrated, which are central to the approach to healthcare navigation and advocacy training. These principles are designed to empower individuals by providing them with the necessary skills to effectively advocate for themselves within the healthcare system, including the guidance that if it does not make sense to you, then it probably does not make sense; don't stop until it makes sense 210, work from the outside in and top to bottom 220, never give up anything once you get it; you will never get it back 230, recognizing that people are people—good, bad, indifferent 240, and emphasizing the importance of research, research, research 250.

The first principle 210, “IF IT DOES NOT MAKE SENSE TO YOU, THEN IT PROBABLY DOES NOT MAKE SENSE; DON′T STOP UNTIL IT MAKES SENSE,” emphasizes the importance of understanding healthcare information. This principle encourages individuals to persist in seeking clarity and comprehension, ensuring that they fully understand the information provided to them. This approach is important for making informed decisions about one's health and care.

The second principle 220, “WORK FROM THE OUTSIDE IN AND TOP TO BOTTOM,” suggests a strategic approach to navigating the healthcare system. This principle advises individuals to start by understanding the broader context and then focus on specific details. This methodical approach helps individuals to effectively manage their healthcare interactions and ensures that they are well-prepared to address any issues that may arise.

The third principle 230, “DO NOT RELINQUISH ANYTHING ONCE YOU OBTAIN SOMETHING; YOU MAY NOT RECOVER IT,” underscores the importance of retaining information and resources. This principle highlights the need to safeguard one's healthcare rights and benefits, ensuring that individuals do not lose access to services or information once they have acquired them.

The fourth principle 240, “PEOPLE ARE PEOPLE—GOOD, BAD, INDIFFERENT,” acknowledges the human element in healthcare interactions. This principle encourages individuals to recognize that healthcare providers, like all people, possess diverse personalities and attitudes. Such understanding can assist individuals in navigating interactions more effectively, fostering improved communication and collaboration.

The fifth principle 250, “RESEARCH, RESEARCH, RESEARCH,” emphasizes the significance of conducting comprehensive research in healthcare advocacy. This principle encourages individuals to actively pursue information, resources, and support to improve their understanding and ability to advocate for themselves. Such an approach empowers individuals to make informed decisions and manage their healthcare needs effectively.

Overall, these principles form a comprehensive framework for self-advocacy, equipping individuals with the skills and strategies necessary to navigate the complexities of the healthcare system, as outlined in the Method 200, which emphasizes that if it does not make sense to you, then it probably does not make sense; don't stop until it makes sense 210, work from the outside in and top to bottom 220, never give up anything once you get it; you will never get it back 230, recognizing that people are people—good, bad, indifferent 240, and the importance of research, research, research 250.

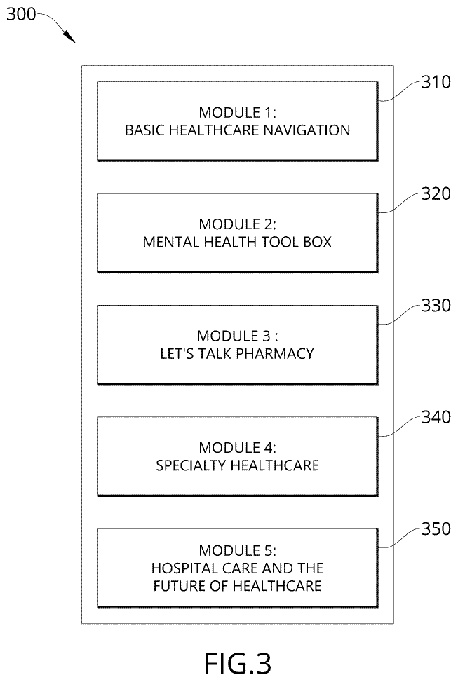

Referring now to FIG. 3 a Learning Modules system 300, which is designed to provide comprehensive training and education in various aspects of healthcare navigation and self-advocacy. This system is composed of five distinct modules, each focusing on a specific area of healthcare knowledge and skills development: MODULE 1: BASIC HEALTHCARE NAVIGATION MODULE 310, MODULE 2: MENTAL HEALTH TOOL BOX MODULE 320, MODULE 3: LET′S TALK PHARMACY MODULE 330, MODULE 4: SPECIALTY HEALTHCARE MODULE 340, and MODULE 5: HOSPITAL CARE AND THE FUTURE OF HEALTHCARE MODULE 350.

Module 1: Basic Healthcare Navigation Module 310 serves as the foundational component of the system 300. The goal is to equip participants with the necessary skills and knowledge required to navigate the healthcare system effectively. This module 310 covers topics such as understanding healthcare terminology, identifying and selecting healthcare providers, and learning how to communicate effectively with healthcare professionals. In embodiments, Module 310 can include at least one anticipation guide, at least one glossary, a plurality of questions, and/or scripts, for utilization by the learner.

In embodiments, the anticipation guide of module 310 is configured to provide both a pre-module and post-module assessment of the learners ability with respect to the module. In embodiments, the anticipation guide of module 310 assesses the leaner on one or more of the following: I can explain what health literacy is to someone else and tell them why it is important to know; I know at least two different ways to contact my doctor; I feel comfortable communicating with my doctor; I know at least 3 questions I could ask my doctor; I can describe what a healthcare transition is and how it works; I can explain how certain body language I see in others might cause me to change how I approach an interaction; I know the difference between a nurse practitioner, physician assistant, and doctor; I am a good self-advocate for my health needs right now; I think this class shares important information that all people should learn; and/or I am interested in learning the other modules in this program.

In embodiments, the one or more questions, or scripts, include one or more questions, for a learner to use in a live, real-time, conversation, such as a phone call, related to topics of Module 310. In embodiments, the one or more questions, or scripts, are dynamically adjustable based on demographic information of the learner. For example, questions can be adjusted based on an age of the learner.

In embodiments, the at least one glossary is configured to provide one or more key terms for the learner, associated with module 310. The at least one glossary can include key terms such as HIPAA, audio/visual body language, elevate, sensory information, health literacy, age out, etc., and are configured to provide knowledge of key terms to the learner.

Module 2: Mental Health Toolbox Module 320 focuses on mental health awareness and management. This module is designed to provide participants with tools and strategies to maintain and improve mental health. The module may include topics such as recognizing mental health issues, understanding mental health resources, and developing coping mechanisms and self-care practices. In embodiments, Module 320 can include at least one anticipation guide, at least one glossary, and at least one coping method. Mental Health Toolbox Module 320 teaches participant(s) to learn mental health coping strategies, how to contact service providers in an emergency, and/or life skills components.

In embodiments, the anticipation guide of module 320 is configured to provide both a pre-module and post-module assessment of the learners ability with respect to the module. In embodiments, the anticipation guide of module 320 assesses the leaner on one or more of the following: I understand how to be a good advocate for my own mental health; I can name one of the three major life skill components to global mental health; I can explain 1 coping strategy and know how I might use it in a stressful situation; I can explain what a nonprofit is to somebody else; I know which nonprofit to call if I am looking for information; I know what nonprofit should I contact when I am having an emergency; and I am interested in learning the next module in this program.

In embodiments, the at least one glossary is configured to provide one or more key terms for the learner, associated with module 320. The at least one glossary can include key terms such as Nonprofit, 211, 988, Warmline Self-Advocacy, Critical Thinking, Interpersonal Communication, Coping Skills, The Folder Method, WHO (World Health Organization), and are configured to provide knowledge of key terms to the learner.

In embodiments, the at least one coping method is the Folder Method. In embodiments, the Folder Method is configured to allow the learner to cope with stress/anxiety inducing situations. In embodiments, the Folder method begins when the learner encounters an issue that is bothering them, at that point the learner writes the issue down, and places the issue in a folder, file, or other container. Once the learner is relaxed, the learner can open the folder and either, deal with one or more issues, or close the folder and try again later. If the learner deals with the issue they can discard it. In embodiments, the process can repeat for any number of issues.

Module 3: Let's Talk Pharmacy Module 330 addresses the role of pharmacies and pharmacists in healthcare. This module educates participants on how to interact with pharmacists, understand medication management, and navigate pharmacy benefits. The module can also cover topics such as understanding prescriptions, over-the-counter medications, and the importance of medication adherence. In embodiments, Module 330 can include at least one anticipation guide, at least one glossary, a plurality of questions, and/or scripts, for utilization by the learner. Let's Talk Pharmacy Module 330 teaches participant(s) to learn crisis management techniques, such as which hotlines to call in differing situations, communication with pharmacists, and/or types of medications and their uses.

In embodiments, the anticipation guide of module 330 is configured to provide both a pre-module and post-module assessment of the learner's ability with respect to the module. In embodiments, the anticipation guide of module 320 assesses the leaner on one or more of the following: I passed on information I learned from earlier sessions to someone I knew; I know which pharmacy my prescriptions are sent to; I know three types of pharmacists or pharmacy types; I know 3 questions I can ask my pharmacist; I understand multiple ways a pharmacist can help me with my healthcare needs; and I know the difference between generic and brand medication.

In embodiments, the at least one glossary is configured to provide one or more key terms for the learner, associated with module 330. The at least one glossary can include key terms such as Pharmacy Benefit Manager (PBM), Buy and Bill, Biosimilar, Step Therapy, Safe Step Act, Protocol, Workaround, and/or The Appeal Process, and are configured to provide knowledge of key terms to the learner.

In embodiments, the one or more questions, or scripts, include one or more questions, for a learner to use in a live, real-time, conversation, such as a phone call, related to topics of Module 330. In embodiments, the one or more questions, or scripts, are dynamically adjustable based on demographic information of the learner. For example, questions can be adjusted based on an age of the learner.

Module 4: Specialty Healthcare Module 340 is dedicated to navigating specialized healthcare services. This module aims to provide participants with the skills needed to access and utilize specialty healthcare providers and services. The module can cover topics such as understanding referrals, selecting specialists, and managing chronic conditions or rare diseases. In embodiments, module 340 can include at least one glossary of key terms, such as healthcare research.

Specialty Healthcare Module 340 teaches participants to learn healthcare navigation techniques, such as how to navigate a hospital, how to communicate with specialty healthcare providers, such as hospitalists, nurses, doctors, etc., and/or how to use their own strengths to communicate with others.

Module 5: Hospital Care and the Future of Healthcare Module 350 focuses on hospital-based care and emerging trends in healthcare. This module covers topics such as understanding hospital systems, preparing for hospital visits, and exploring future healthcare innovations. The module can also address the integration of technology in healthcare and the evolving landscape of patient care. In embodiments, Module 350 can include at least one anticipation guide, at least one glossary, a plurality of questions, and/or scripts, for utilization by the learner.

Hospital Care and the Future of Healthcare Module 350 participant(s) learn principles of self-advocacy 200, roles and responsibilities of different hospital employees, and the importance of asking the right questions in hospitals.

In embodiments, the anticipation guide of module 350 is configured to provide both a pre-module and post-module assessment of the learner's ability with respect to the module. In embodiments, the anticipation guide of module 350 assesses the leaner on one or more of the following: Name two questions that you should ask every hospital you call; Explain why everyone should call Patient Financial Services and two types of information they can help you with; and/or Describe the role of the social worker in a hospital; Identify one of the five principles of advocating as it relates to your mental and physical health.

In embodiments, the one or more questions, or scripts, include one or more questions, for a learner to use in a live, real-time, conversation, such as a phone call, related to topics of Module 350. In embodiments, the one or more questions, or scripts, are dynamically adjustable based on demographic information of the learner. For example, questions can be adjusted based on an age of the learner.

In embodiments, the at least one glossary is configured to provide one or more key terms for the learner, associated with module 350. The at least one glossary can include key terms such as Bucket System, Hospital Community Outreach, Clinical Pharmacist, Patient Financial Counseling, and/or The Five Principles of Self-Advocacy

Overall, the Learning Modules system 300 is designed to provide a comprehensive educational framework that empowers individuals to become informed and proactive participants in their healthcare journey. Each module builds upon the previous one, creating a cohesive and integrated learning experience that addresses the diverse needs of healthcare consumers, including MODULE 1: BASIC HEALTHCARE NAVIGATION 310, MODULE 2: MENTAL HEALTH TOOLBOX 320, MODULE 3: LET′S TALK PHARMACY 330, MODULE 4: SPECIALTY HEALTHCARE 340, and MODULE 5: HOSPITAL CARE AND THE FUTURE OF HEALTHCARE 350.

The present invention can be based on storytelling in an active learning environment (discussion, practice, teach another retain 90%) include the life skills communication, decision-making, and coping strategies. The present invention includes live calls with participants to get buy-in and take away fear and everything customized to the participants. The present invention incorporates Five Principles of Self-Advocacy and can work as a whole or in pieces and tailored for beginner, intermediate, advanced. The present invention can be implemented in various environments, for example, youth transitioning from pediatric to adult care, employee health and wellness, rare disease/disability community, criminal justice system/rehab to break the revolving door by giving people a way to self-advocate for themselves that is private and empowering, university freshman program, taught as train the trainer for medical school, doctor of occupational therapy, social workers, and the like. The present invention can be used with various populations such as aged populations, indigenous, and rural communities. All skills learned during the training and learning method are transferable to other parts of life.

The present invention is based on Five Principles of Self-Advocacy and woven into the entire process. The principles can include People are People, Don't stop until it makes sense, work from the outside in and top to bottom, never give up anything; you will never ever get it back, Research, Research, Research. The present invention can be integrated into basic healthcare navigation. For youth transitioning to adult care, the training and learning method can teach transitioning to adult care, body language and telephone skills, at what age we will be transitioning to adult care, what is the difference between a doctor, nurse practitioner, and physician assistant, and what HIPAA compliant questions we should ask of any doctor's office. The present invention can finish by calling physicians together live on speaker.

For adults, the present invention can teach body language and telephone skills, differences between doctors, nurse practitioners, and physician assistants, what HIPAA-compliant questions we can ask a doctor's office to make sure they are correctly fit and will advocate for you. The present invention can finish by calling physicians together live on speaker.

The present invention can include a Mental Health Toolbox component that can be paired with The Five Principles of Self-Advocacy or as part of a process design based on participants. Mental Health talks about Three main categories of life skills needed for global mental health and gives live examples, role play of thinking and decision-making interpersonal/communication, coping and self-management and ends with a live call to a trusted and confidential resource open 24/7.

The present invention can include Let's Talk Pharmacy components that can be stand along with Five Principles of Self-Advocacy. The Let's Talk Pharmacy components talks about your Pharmacist being the Doctor of your medication and free and some open 24/7. The Let's Talk Pharmacy components provides instruction about the different types of pharmacies and pharmacists and then make live calls using a HIPAA compliant questions to pharmacies together. If for rare disease, chronic illness, seniors, adults can cover step therapy, overrides, workarounds, carve-out, carve-in, Pharmacy Benefit Managers.

The present invention can include a Specialty Healthcare component. This component provides instruction about how to research new doctors and interview them using trusted resources. This component provides instruction to research hospitals as well as new internists and specialists.

The present invention can include Hospital Care & The Future of Healthcare component. This component includes calling the hospitals we researched and speak with Patient Financial Services, the Hospital Social Worker and the Clinical Pharmacist on speaker to get information before we need it instead of after when it's too late.

The present invention can include a Financial Literacy component. This component includes calling our bankers and asking questions to understand the differences between a credit union, savings and loan, local bank vs large system. The training and learning method can include a gender equality component.

The present invention scales/works through a Train the Trainer Model. The Train the Trainer can be performed manually and can be embodied in an application where be collected to use for measuring data collected. In this example, the live calls would be made from the application.

The present invention works By learning the Five Principles of Self-Advocacy through storytelling, live examples that allows participants to understand by actually doing it themselves what it means to think differently/outside the box learning about understanding body language both audio and visual, to understand different personalities, phone etiquette, learning together and doing everything together in a interactive and immersive environment not being distracted. Once they learn the modules they can teach each one to seven people and then become an ambassador of our program and be trained as trainer.

In some embodiments, methods and modules of the present invention, with exception of the Five Principles of Self-Advocacy, are mix-and-match and can be shuffled, interchanged or reconfigured based on the participants needs and skill-set and taught to a group or teach a group to be trainers.

In one example, a pharmacist could be trained in the training and learning method 100 and then bring the program into their communities where there are cures for rare diseases if only everyone knew to contact their local pharmacist with weird symptoms. They have databases they can search and input into. They also can diagnose certain illnesses and give vaccines. They are free and before going to any Emergency Room you should be taught to call the free pharmacist first. Training local pharmacists and in doctor of pharmacy program will allow our pharmacists to be the center of their communities which will make a huge difference in buy-in and save lives and money.

In another example, the training and learning method 100 can be used to train the Social Worker and Nurse Practitioner as trainers to give to kids as needed as a one-on-one or as groups.

Any of the aspects of the training and learning method can be performed using a computing device storing and executing an application to assist with the training and learning method. In embodiments, a computing device can include one or more electronic devices such as a laptop computer, a desktop computer, a tablet computer, a smartphone, a thin client, and the like. The computing device an include a processing device coupled to a communication device. The processing device is also coupled to a memory device, and an input/output (“I/O”) interface. In embodiments, the communication interface enables the computing device to communicate with other devices and systems via one or more networks.

The processing device, the communication device, the memory device, and the I/O interface can be interconnected via a system bus. The system bus can be and/or include a control bus, a data bus, an address bus, and the like. The processing device can be and/or include a processor, a microprocessor, a computer processing unit (“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and the like. As used herein, “processor,” “processing component,” “processing device,” and/or “processing unit” can be used generically to refer to any or all of the aforementioned specific devices, elements, and/or features of the processing device.

The memory device can be and/or include one or more computerized storage media capable of storing electronic data temporarily, semi-permanently, or permanently. The memory device can be or include a computer processing unit register, a cache memory, a magnetic disk, an optical disk, a solid-state drive, and the like. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device.

The communication device enables the computing device to communicate with other devices and systems. The communication device can include hardware and/or software for generating and communicating signals over a direct and/or indirect network communication link. As used herein, a direct link can include a link between two devices where information is communicated from one device to the other without passing through an intermediary. For example, the direct link can include a Bluetooth™M connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. programming installed on a processor, such as the processing component, coupled to the antenna.

An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

In embodiments, the components and functionality of the computing device can be hosted and/or instantiated on a “cloud” and/or “cloud service.” As used herein, a “cloud” and/or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

The computing device can include a user interface for outputting information in a format perceptible by a user and receiving input from the user. The user interface can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The user interface can include an acoustic element such as a speaker, a microphone, and so forth. The user interface can include a button, a switch, a keyboard, a touch-sensitive surface, a touchscreen, a camera, a fingerprint scanner, and so forth. The touchscreen can include a resistive touchscreen, a capacitive touchscreen, and so forth.

In embodiments, an interactive game component can be included as a component of the system. In embodiments, the interactive game component can be an application, module, software, or program, running on a computing device, and configured to promote understanding and retention of the system of the present invention.

In embodiments, the interactive game component can include one or more user interface elements, such as a player, and one or more gaming elements. In embodiments, the player can be controlled to move throughout an environment, using one or more user interfaces, such as a keyboard, mouse, etc., to interact with the one or more gaming elements.

The one or more gaming elements can include a obstacle elements, and/or objective elements. In embodiments, the obstacle elements can include one or more icons, such as apple cores, configured to negatively impact the player, such as by taking away points, or lives. In embodiments, objective elements, such as medical packs, transition the player to a question an answer screen where one or more questions regarding one or more aspects of the system are presented to the user to answer. In embodiments, a correct answer is configured to positively impact the player, such as by adding points, and/or lives. Advantageously, the interactive game component allows for gamification of the system such that retention is improved.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

What is claimed is:

1. A training and self-advocacy a user method, comprising:
providing, to the user, one or more narratives;
performing, by the user, one or more learning modules configured to teach one or more outcomes;
performing, by the user, one or more real-world exercises based on the one or more learning modules; and
sharing, by the user, the one or more outcomes to at least one additional user.

2. The method of claim 1, wherein the one or more real-word exercises is a live telephone conversation with a person identified in the one or more learning modules.

3. The method of claim 1, wherein the one or more learning modules include at least one of:
a basic healthcare navigation module configured to equip participants with one or more skills or knowledge to navigate the healthcare system;
a mental health toolbox module configured to provide participants with tools and strategies to maintain or improve mental health;
a pharmacy education module configured to educate the user on how to interact with pharmacists, understand medication management, and navigate pharmacy benefits;
a specialty healthcare module configured to provide the user with one or more skills to access and utilize specialty healthcare providers or services; or
an advanced healthcare navigation module configured to educate the user on hospital systems, preparing for hospital visits, and exploring future healthcare innovations.

4. The method of claim 3, wherein each of the one or more learning modules include at least one of: an anticipation guide, a glossary, one or more scripts, and/or one or more additional learning resources.

5. The method of claim 4, wherein the one or more scripts is dynamically adjustable based on one or more demographic information of the user.

6. A training and self-advocacy game, comprising:
at least one processor, and at least one memory, the memory storing instructions that when executed cause the processor to perform a method, the method comprising:
receiving, by a user interface, a command to navigate a player in a gaming environment;
moving, in response to the command, the player in the gaming environment;
determining, in response to an interaction by the player with one or more gaming elements, whether the one or more gaming elements is an objective gaming element, or an obstacle gaming element;
in response to the one or more gaming element being an objective gaming element, presenting one or more questions to the user, wherein the one or more questions reinforce information provided in one or more learning modules;
and
in response to the one or more gaming element being an obstacle gaming element, decrementing a life element of the player.
